(19) United States
(12) Patent Application Publication
(10) Pub. No.: US 2018/0339118 A1
(43) Pub. Date: Nov. 29, 2018

(54) ELECTRONIC CIGARETTE

(71) Applicant: Shenzhen First Union Technology Co., Ltd., Shenzhen (CN)

(72) Inventors: Zhi OUYANG, Shenzhen (CN); Meng LI, Shenzhen (CN); Huiyun CHEN, Shenzhen (CN); Zhongli XU, Shenzhen (CN); Yonghai LI, Shenzhen (CN)

(73) Assignee: Shenzhen First Union Technology Co., Ltd., Shenzhen (CN)

(21) Appl. No.: 15/872,947

(22) Filed: Jan. 16, 2018

(30) Foreign Application Priority Data
May 25, 2017 (CN) 201720590897.7

(51) Int. Cl. A61M 15/00; A24F 47/00; A61M 15/06; A61M 11/04; A24D 3/18

(52) U.S. Cl. CPC A61M 15/0025; A24F 47/008; A24D 3/18; A61M 11/042; A61M 15/06

(57) ABSTRACT

The present disclosure provides an electronic cigarette having an invertible mouthpiece. The electronic cigarette includes a battery assembly and an atomization assembly, the battery assembly including a battery and a housing, and the atomization assembly including a mouthpiece, an outer sleeve and a cartridge. The atomization assembly and the battery assembly are in pluggable connection. The housing defines a groove in the upper part thereof. The outer sleeve has a tail end accommodated into the groove in a working state. The atomization assembly is inverted and the mouthpiece is accommodated into the groove in a receiving state. After the electronic cigarette is used, the mouthpiece can be inverted conveniently and can be completely hidden in the housing to be protected against contamination.

### CROSS REFERENCE TO RELATED APPLICATIONS

The present application claims priority to Chinese patent application CN 201720590897.7 filed on May 25, 2017.

### TECHNICAL FIELD

The present disclosure relates to the field of electronic cigarettes, and particularly, to an electronic cigarette having an invertible mouthpiece.

### BACKGROUND

Tobacco smoke contains dozens of carcinogens (for example, tar), which are believed to have a great harm to human health. Furthermore, the smoke hangs in air, making the surrounding people passive to smoke and causing damages to their bodies. Therefore, most public places forbid smoking expressly. In order to meet the requirements of part smokers, electronic cigarette appears accordingly.

At present, most electronic cigarettes available on the market have the mouthpiece exposed to outside. The mouthpiece is easy to be contaminated by dust, oil, etc., thereby influencing the experience of consumers. A few electronic cigarettes can be put in a storage box or accommodation box entirely after use. However, configuring a storage box or accommodation box separately increases the cost, and sometimes is inconvenient.

For example, a Chinese patent with application number of CN201620403775.8 discloses a mouthpiece and an electronic cigarette thereof, in which the mouthpiece can be stored in a storage groove in a rotatable manner, so that the external contamination to the mouthpiece is reduced. As shown in FIG. 1, after the mouthpiece is stored, the inhaling end is still partially exposed to outside. It's not possible to completely avoid contamination. Another Chinese patent with application number of CN201420654658.X discloses a flue-cured type electronic cigarette having a telescopic mouthpiece, in which, one end of a guide rail is connected to a mouthpiece, and a housing defines therein a movement groove allowing the guide rail to move up down. Such arrangement can avoid the condition that the mouthpiece is over protruded from the surface of the electronic cigarette during the non-use state. However, this scheme also cannot completely prevent the mouthpiece being contaminated.

### SUMMARY

The technical problem to be solved by the present disclosure is to remedy the drawbacks of the prior art by providing an electronic cigarette which is capable of completely hiding a mouthpiece, and is convenient to operate and low in cost.

In order to solve the above technical problem, the present disclosure employs a following technical scheme. An electronic cigarette having an invertible mouthpiece includes a battery assembly and an atomization assembly, the battery assembly including a battery and a housing, and the atomization assembly including a mouthpiece, an outer sleeve and a cartridge. The atomization assembly and the battery assembly are in pluggable connection. The housing defines a groove in the upper part thereof. The outer sleeve has a tail end accommodated into the groove in a working state, and the atomization assembly is inverted and the mouthpiece is accommodated into the groove in a receiving state.

The outer sleeve has an outer surface matched with the housing in shape and size.

The mouthpiece and the outer sleeve of the atomization assembly are in an integrated structure or detachable structure.

The cartridge and the outer sleeve are in an integrated structure.

The cartridge and the outer sleeve are in a detachable structure.

The outer sleeve defines a vertical thin slot on a lateral edge thereof, the cartridge is provided with a convex column on the outside, the position of the convex column corresponds to position of the thin slot, and the convex column protrudes from the outer surface of the outer sleeve.

The number of the thin slot is two, and the number of the convex column is two too.

The outer sleeve is provided with a bottom cap; the bottom cap and the mouthpiece are disposed at two ends of the outer sleeve respectively.

The outer sleeve defines a vertical vision window.

The beneficial effects of the present disclosure are as follows. The atomization assembly and the battery assembly are in pluggable connection, thus it is convenient and labor saving to invert the mouthpiece. In each of the working state and the receiving state, the outer surface of the outer sleeve fully fits the upper part of the housing, thereby achieving a general dustproof effect, saving space for the user and protecting the user's privacy properly.

### BRIEF DESCRIPTION OF THE DRAWINGS

FIG. 1 is a cross-sectional view of an electronic cigarette in a working state.

FIG. 2 is a cross-sectional view of an electronic cigarette in a receiving state.

FIG. 3 is a perspective view of an electronic cigarette in a working state.

FIG. 4 is a perspective view of an electronic cigarette in a receiving state.

FIG. 5 is a side view of an electronic cigarette in a working state.

FIG. 6 is a side view of an electronic cigarette in an intermediate state.

FIG. 7 is a side view of an electronic cigarette in a receiving state.

FIG. 8 is a perspective view of a second embodiment of an electronic cigarette in a working state.

FIG. 9 is a perspective view of a second embodiment of an electronic cigarette in a receiving state.

FIG. 10 is an exploded view of a second embodiment of an electronic cigarette in a receiving state.

FIG. 11 is a cross-sectional view of a third embodiment of an electronic cigarette in a working state.

FIG. 12 is an exploded view of a third embodiment of an electronic cigarette in a working state.

FIG. 13 is an exploded view of a fourth embodiment of an electronic cigarette in a working state.

### DETAILED DESCRIPTION

An electronic cigarette having an invertible mouthpiece provided by the present disclosure is described below in further detail in conjunction with the drawings.

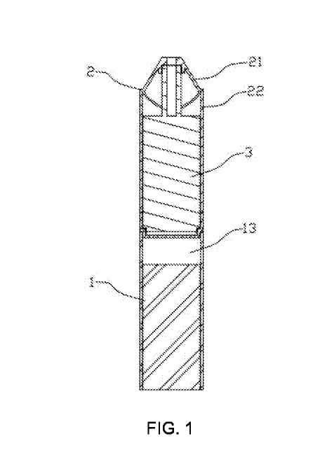

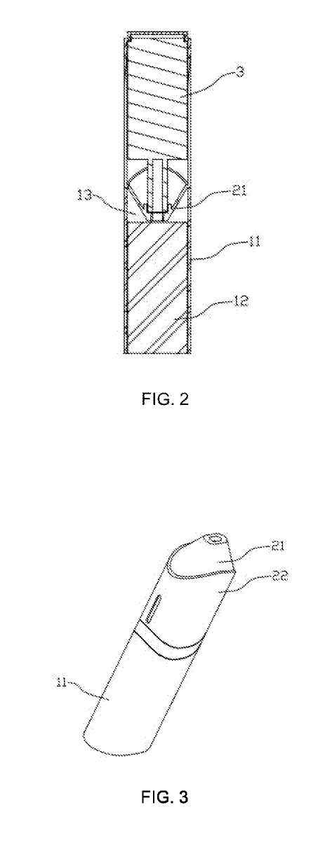

FIG. 1 to FIG. 7 illustrates a first embodiment of an electronic cigarette. The electronic cigarette includes a battery assembly 1 and an atomization assembly 2 that are coupled together. The battery assembly 1 includes a housing 11, and a battery 12 built in the housing. The atomization assembly 2 includes a mouthpiece 21 and an outer sleeve 22. The mouthpiece 21 is located at an upper end of outer sleeve 22.

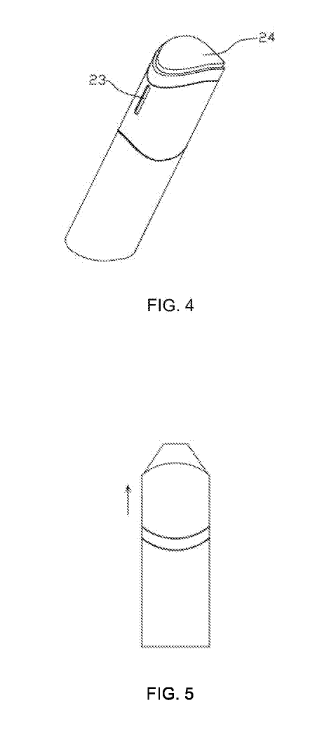

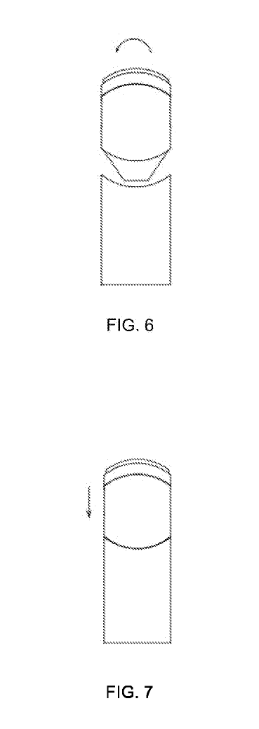

The housing 11 of the battery assembly 1 defines a groove 13 in the upper part thereof. In a working state, as shown in FIG. 1, FIG. 3 and FIG. 5, the outer sleeve 22 has a tail end accommodated into the groove 13. The outer sleeve 22 has an outer surface matched with the housing 11 in shape and size. After the electronic cigarette is used, during the storage process, as shown in FIG. 5, the atomization assembly 2 is lifted upwards and pulled out entirely, so that the tail end of the atomization assembly 2 is disengaged from the groove 13; then, as shown in FIG. 6, the atomization assembly 2 is overturned downwards by 180 degrees entirely; next, as shown in FIG. 7, the atomization assembly 2 is inserted downwards, so that the mouthpiece 21 of the atomization assembly 2 is completely accommodated into the groove 13. The working state is as shown in FIG. 3, and the receiving state is as shown in FIG. 4. The outer surface of the outer sleeve 22 fully fits the upper part of the housing 11, thereby being capable of preventing entrance of water and dust. In this way, the mouthpiece 21 is stored by simple and quick pullout and insertion, without extra parts. This arrangement not only achieves a general dustproof effect, but also saves space for the user and protects the user's privacy properly. When the atomization assembly 2 is operated from the receiving state to the working state, the operation is just the opposite, and no further description is needed here.

In the first embodiment, the atomization assembly 2 further has a cartridge 3 disposed therein. The cartridge 3 can contain tobacco liquid, tobacco paste, solid tobacco material, etc. The cartridge 3 and the outer sleeve 22 are fixed through an integrated structure. The mouthpiece 21 and the outer sleeve 22 are in an integrated structure, that is, the mouthpiece 21 and the outer sleeve 22 are not detachable from each other. The outer sleeve 22 further defines a vision window 23. The vision window 23 is defined vertically, so that the user can check the rest of the cartridge 3 at any time. The outer sleeve 22 is further provided with a bottom cap 24. The bottom cap 24 and the mouthpiece 21 are disposed at two ends of the outer sleeve 22 respectively.

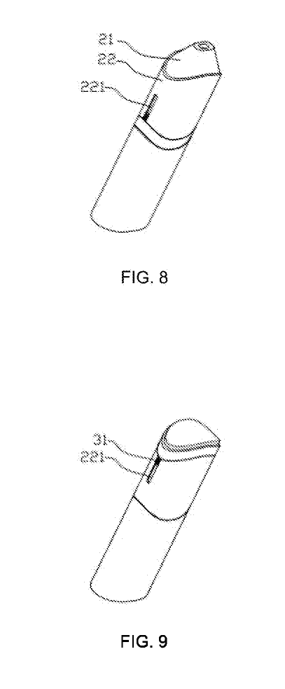

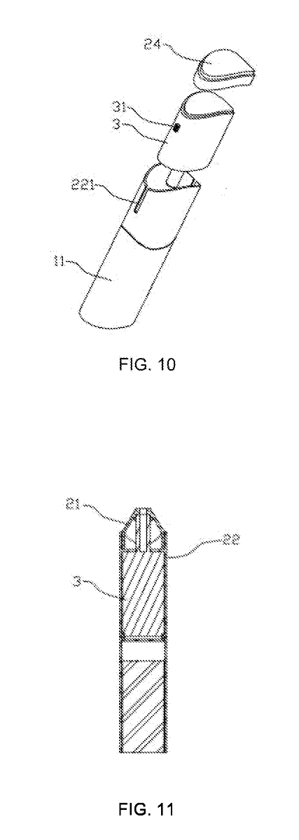

A second embodiment of the electronic cigarette is as shown in FIG. 8 to FIG. 10. The present embodiment has most of the structure the same as the first embodiment, with a difference lying that the cartridge 3 and the outer sleeve 22 are in a detachable structure. Such arrangement has the purposes that an exhausted cartridge 3 can be replaced and that the outer sleeve 22 and the mouthpiece 21 can be reused to save consumption of materials. In the second embodiment, the outer sleeve 22 defines a vertical thin slot 221 on a lateral edge thereof, the cartridge 3 is provided with a convex column 31 on the outside, the position of the convex column 31 corresponds to position of the thin slot 221, and the convex column 31 slightly protrudes from the outer surface of the outer sleeve 22. When the cartridge 3 is put into the outer sleeve 22, the convex column 31 slides along the thin slot 221, thereby ensuring the successful assembly and ensuring the cartridge 3 to be stable and not to swing in the outer sleeve 22. After the cartridge 3 is assembled, the bottom cap 24 is mounted. Then, the atomization assembly 2 is overturned entirely, so that the mouthpiece 21 is disposed at the upper end. Thus, the working state is ready.

In the second embodiment, the number of the thin slot 221 is two preferably, and the number of the convex column 31 is two too. That is, the thin slots 221 are defined on two lateral edges of the outer sleeve 22 symmetrically. Such arrangement has advantages that, no matter the cartridge 3 is put into or removed from the outer sleeve 22, the cartridge 3 can be moved by clamping the convex columns 31 with two fingers of a single hand respectively. Another purpose of the thin slot 221 lies in that the user can view the usage and consumption condition of the cartridge 3 through the thin slot 221, so as to replace the cartridge 3 in time and prevent dry-burning.

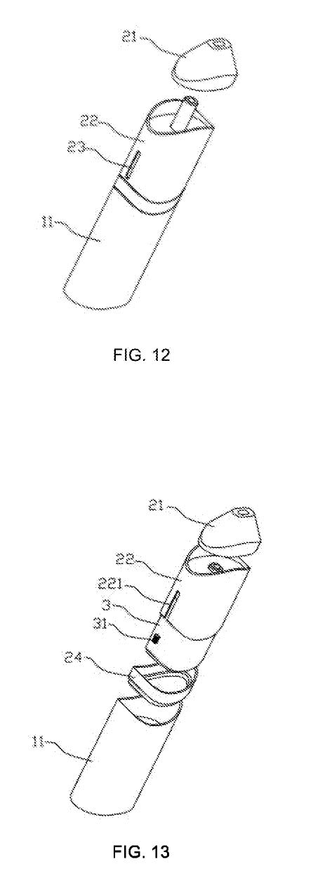

A third embodiment of the electronic cigarette is as shown in FIG. 11 to FIG. 12. The present embodiment has most of the structure the same as the first embodiment, with differences lying that the mouthpiece 21 and the outer sleeve 22 are in a detachable structure, the mouthpiece 21 has a tail end accommodated into a groove on the upper end of the outer sleeve 22, and the mouthpiece 21 and the outer sleeve 22 are in pluggable connection. Such arrangement has the purpose that the user can replace the mouthpiece with different shapes of mouthpieces according to the personal habit, or that the mouthpiece can be replaced separately when aged or accidentally damaged or contaminated, without the replacement of the outer sleeve 22, so that the cost is saved and the selectivity of the user is increased.

A fourth embodiment of the electronic cigarette is as shown in FIG. 13. The present embodiment has most of the structure the same as the first embodiment, with differences lying that the cartridge 3 and the outer sleeve 22 are in a detachable structure and the mouthpiece 21 and the outer sleeve 22 are in a detachable structure.

The above embodiments are merely partial implementations listed in the description to help understand the content of the present disclosure, and they neither restrict the technical scheme of the present disclosure, nor make an exhaustion of all schemes implementable. Any minor improvements or equivalent substitutions made to the structures, processes or steps of the present disclosure are intended to be included in the scope of protection of the present disclosure.

What is claimed is:

1. An electronic cigarette, comprising a battery assembly and an atomization assembly, the battery assembly comprising a battery and a housing, and the atomization assembly comprising a mouthpiece, an outer sleeve and a cartridge, wherein the atomization assembly and the battery assembly are in pluggable connection, the housing defines a groove in the upper part thereof, the outer sleeve has a tail end accommodated into the groove in a working state, and the atomization assembly is inverted and the mouthpiece is accommodated into the groove in a receiving state.

2. The electronic cigarette according to claim 1, wherein the outer sleeve has an outer surface matched with the housing in shape and size.

3. The electronic cigarette according to claim 1, wherein the mouthpiece and the outer sleeve of the atomization assembly are in an integrated structure or a detachable structure.

4. The electronic cigarette according to claim 1, wherein the cartridge and the outer sleeve are in an integrated structure.

5. The electronic cigarette according to claim 1, wherein the cartridge and the outer sleeve are in a detachable structure.

6. The electronic cigarette according to claim 5, wherein the outer sleeve defines a vertical thin slot on a lateral edge thereof, the cartridge is provided with a convex column on the outside, the position of the convex column corresponds to position of the thin slot, and the convex column protrudes from the outer surface of the outer sleeve.

7. The electronic cigarette according to claim 6, wherein the number of the thin slot is two, and the number of the convex column is two too.

8. The electronic cigarette according to claim 1, wherein the outer sleeve is provided with a bottom cap; the bottom cap and the mouthpiece are disposed at two ends of the outer sleeve respectively.

9. The electronic cigarette according to claim 1, wherein the outer sleeve defines a vertical vision window.
